(19) United States
(12) Patent Application Publication
(10) Pub. No.: US 2013/0096189 A1
(43) Pub. Date: Apr. 18, 2013

(54) USE OF GLABRANIN FOR STIMULATING HAIR GROWTH

(71) Applicants: Ranjit Kaur Bhogal, Sharnbrook (GB); Julia Sarah Rogers, Sharnbrook (GB)

(72) Inventors: Ranjit Kaur Bhogal, Sharnbrook (GB); Julia Sarah Rogers, Sharnbrook (GB)

(21) Appl. No.: 13/704,996

(22) PCT Filed: Jul. 4, 2011
(86) PCT No.: PCT/EP2011/061243

§ 371 Date: Dec. 18, 2012

(30) Foreign Application Priority Data
Jul. 6, 2010 (EP) 10168504.8

(51) Int. Cl. A61K 8/49; A61Q 7/00

(52) U.S. Cl. CPC A61K 8/498; A61Q 7/00
USPC 514/456; 549/403

(57) ABSTRACT

One of the major hair concerns for both men and women is that of hair loss. For men the most obvious manifestation of this is male pattern baldness known as alopecia areata. However hair shedding resulting in hair thinning is also a concern for both men and women. Individuals suffering hair loss often experience psychological disadvantages, for example social phobia, anxiety, and depression, due to their change in appearance. This invention relates to a hair care composition comprising glabranin or a derivative thereof. The invention also relates to a cosmetic method for increasing hair fibre diameter, stimulating hair growth, retaining hair or reducing hair loss, stimulating hair follicle growth in anagen or telogen phases, increasing hair fibre density and preventing or treating alopecia.

This invention relates to a hair care composition comprising glabranin or a derivative thereof. The invention also relates to a cosmetic method for increasing hair fibre diameter, stimulating hair growth, retaining hair or reducing hair loss, stimulating hair follicle growth in anagen or telogen phases, increasing hair fibre density and preventing or treating alopecia.

One of the major hair concerns for both men and women is that of hair loss. For men the most obvious manifestation of this is male pattern baldness known as alopecia areata. However hair shedding resulting in hair thinning is also a concern for both men and women. Individuals suffering hair loss often experience psychological disadvantages, for example social phobia, anxiety, and depression, due to their change in appearance.

Various attempts have been made to combat these problems. For example stress reduction can be helpful in slowing hair loss (as stress can often be linked to periods of hair loss). A number of drugs, operating in quite different ways, have been used extensively. For example immunosuppressants applied to the scalp have been shown to temporarily reverse alopecia areata although there can be significant side effects from this treatment. Use of dihydrotestosterone inhibitors such as finasteride (in the form of an oral intervention) has been approved and 5-alpha reductase inhibitors such as ketoconazole have also been recommended. Other drugs include potassium channel opening and vasodilator compounds such as minoxidil (in the form of a 2% w/w topical solution) and diazoxide.

Minoxidil was originally produced as an oral anti-hypertensive drug (Loniten™), which was found to also have hypertrichosis effects. Messenger et al (Br. J. Dermatol., 150/2, 186-194 (2004)), showed in clinical trials that topical minoxidil increases hair growth and diameter, triggering anagen induction and prolonging length of anagen and hence hair. In terms of the mode-of-action, Sanders et al (J. Invest. Dermatol., 107/2, 229-234 (1996)) have shown that minoxidil is able to increase cell proliferation in NIH 3T3 fibroblasts via the opening of potassium channels. Messenger et al showed that other potassium channel openers exhibit a similar hypertrichosis effect. Lachgar et al (Br. J. Dermatol., 138/3,407-411 (1998)) has shown that minoxidil increases the vascular endothelial growth factor (VEGF) which plays an important role in hair growth angiogenesis and follicle cycling. In dermal papilla cells, Li et al (J. Invest Dermatol., 117/6, 1594-1600 (2001)) has shown that minoxidil binds to the sulphonylurea receptor SUR2B component of KATP channels resulting in the secretion of ATP which is converted to adenosine and results in an increase VEGF levels.

The side effects of minoxidil include itchy scalp and dandruff, often due to the alcohol required for its topical formulation. Side effects of oral minoxidil include swelling of the face and extremities, rapid and irregular heartbeat, lightheadedness and cardiac lesions. Herbal treatments with saw palmetto and green tea extracts (both of which comprise 5-alpha reductase inhibitors), and Gingko biloba extract (which comprises an active vasodilator) have been used. Non-invasive treatments include scalp massage with essential oils. Caffeine has also been identified as a stimulator of human hair growth in-vitro, and reduced testosterone-induced follicle growth suppression.

An in-vitro screening assay to identify potassium channel openers through measuring cell mitogenesis in low serum conditions, in the absence of aminoglycoside antibiotics has been described previously in EP 0 842 427 which confirms the involvement of the potassium channel in the mitogenic effects via the use of multiple potassium channel blockers, more specifically KATP specific antagonists, such as tolbutamide or glibenclamide, in combination with weak non-selective potassium channel pore blockers such as tetraethylammonium (TEA).

### SUMMARY OF THE INVENTION

In a first aspect of the invention a solution to the aforementioned problem of hair loss is provided in the form of a hair care composition comprising glabranin or a derivative thereof. Glabranin is a flavone of empirical chemical formula C₂₀H₂₀O₄ which, according to Hayashi et al. (Chem. Pharm. Bull., 51/11, 1338-1340 (2003)) can be sourced from the leaves and shoots, but not the root, of the liquorice plant, from Annona glabra seeds and from Tephrosia madrensis. The IUPAC name for glabranin is (2S)-5,7-dihydroxy-8-(3-methylbut-2-enyl)-2-phenyl-2,3-dihydrochromen-4-one and has the following chemical structure:

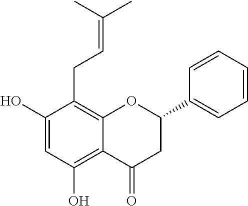 [SMILES: CC(C)=CCc1c(O)cc(O)c2c1O[C@H](c1ccccc1)CC2=O]

The glabranin or a derivative thereof may be in the form of an extract of any one of the group consisting of a leaf of Glycyrrhiza glabra, a seed of Annona glabra, Tephrosia species such as Tephrosia madrensis, Esenbeckia species such as Esenbeckia berlandieri, and Annona squamosa. Preferably the glabranin or a derivative thereof is in the weight range 0.00001 to 30%, preferably 0.0001 to 15%, most preferably 0.001 to 5% by weight of the composition.

Derivatives thereof include methyl and acetyl derivatives as well as glucosides.

In a second aspect of the invention, glabranin or a derivative thereof is provided for use in increasing the proliferation of cells within a hair follicle. In the alternative a method for increasing the proliferation of cells within a hair follicle is provided, the method comprising the step of providing to a person in need thereof an effective amount of glabranin or a derivative thereof.

In a third aspect of the invention, a cosmetic method for increasing hair fibre diameter is provided, the method comprising the step of applying the hair care composition of the first aspect of the invention to a scalp or hair of a person in need thereof.

In a fourth aspect of the invention, a cosmetic method for stimulating hair growth is provided, the method comprising the step of applying the hair care composition of the first aspect of the invention to a scalp or hair of a person in need thereof.

In an fifth aspect of the invention, a cosmetic method for retaining hair or reducing hair loss is provided, the method the step of comprising applying the hair care composition of the first aspect of the invention to a scalp or hair of a person in need thereof.

In a sixth aspect of the invention, a cosmetic method for stimulating anagen induction is provided, the method comprising the step of applying the hair care composition of the first aspect of the invention to a scalp or hair of a person in need thereof.

In a seventh aspect of the invention, a cosmetic method for increasing hair fibre density is provided, the method comprising the step of applying the hair care composition of the first aspect of the invention to a scalp or hair of a person in need thereof.

In an eighth aspect of the invention, a cosmetic method for preventing or treating alopecia is provided, the method comprising the step of applying the hair care composition of the first aspect of the invention to a scalp or hair of a person in need thereof.

### DETAILED DESCRIPTION OF THE INVENTION

The final product form of the hair care compositions according to the invention may suitably be, for example, shampoos, conditioners, sprays, mousses, gels, oils, creams, waxes or lotions. Particularly preferred product forms are leave-in products, especially post-wash conditioners (leave-in) and hair treatment products such as hair essences.

Conditioning Surfactant

Conditioner compositions usually comprise one or more conditioning surfactants, which are cosmetically acceptable and suitable for topical application to the hair.

Suitable conditioning surfactants are selected from cationic surfactants, used singly or in a mixture.

Cationic surfactants useful in compositions of the invention contain amino or quaternary ammonium hydrophilic moieties, which are positively charged when, dissolved in the aqueous composition of the present invention.

The most preferred cationic surfactants for conditioner compositions of the present invention are monoalkyl quaternary ammonium compounds in which the alkyl chain length is C16 to C22.

Examples of suitable cationic surfactants include quaternary ammonium compounds, particularly trimethyl quaternary compounds.

Preferred quaternary ammonium compounds include cetyltrimethylammonium chloride, behenyltrimethylammonium chloride (BTAC), cetylpyridinium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, cocotrimethylammonium chloride, PEG-2 oleylammonium chloride and salts of these where the chloride is replaced by halogen, (e.g. , bromide), acetate, citrate, lactate, glycolate, phosphate nitrate, sulphate, or alkylsulphate. Further suitable cationic surfactants include those materials having the CTFA designations Quaternium-5, Quaternium-31 and Quaternium-18. Mixtures of any of the foregoing materials may also be suitable. A particularly useful cationic surfactant for use in hair conditioners of the invention is cetyltrimethylammonium chloride, available commercially, for example as GENAMIN CTAC, ex Hoechst Celanese.

Salts of primary, secondary, and tertiary fatty amines are also suitable cationic surfactants.

In the conditioners of the invention, the level of cationic surfactant is preferably from 0.01 to 10, more preferably 0.05 to 5, most preferably 0.1 to 2% w/w of the total composition.

Fatty Materials

Conditioner compositions of the invention preferably additionally comprise fatty materials. By “fatty material” is meant a fatty alcohol, an alkoxylated fatty alcohol, a fatty acid, a glyceride, glycerol, plant unsaponifiables or a mixture thereof.

Representative fatty alcohols comprise from 8 to 22 carbon atoms, more preferably 16 to 22. Fatty alcohols are typically compounds containing straight chain alkyl groups. Examples of suitable fatty alcohols include cetyl alcohol, stearyl alcohol and mixtures thereof. The use of these materials is also advantageous in that they contribute to the overall conditioning properties of the composition.

Ethoxylated fatty alcohols having from about 12 to about 18 carbon atoms in the alkyl chain can be used in place of, or in addition to, the fatty alcohols themselves.

Suitable examples include ethylene glycol cetyl ether, polyoxyethylene (2) stearyl ether, polyoxyethylene (4) cetyl ether, and mixtures thereof.

The level of fatty alcohol material in conditioners of the invention is suitably from 0.01 to 15, preferably from 0.1 to 10, and more preferably from 0.1 to 5% w/w.

The weight ratio of cationic surfactant to fatty alcohol is suitably from 10:1 to 1:10, preferably from 4:1 to 1:8, optimally from 1:1 to 1:7, for example 1:3.

Suspending Agents

In a preferred embodiment, the hair care compositions, especially if it is a shampoo, further comprises from 0.1 to 5% w/w of a suspending agent.

Suitable suspending agents are selected from polyacrylic acids, cross-linked polymers of acrylic acid, copolymers of acrylic acid with a hydrophobic monomer, copolymers of carboxylic acid-containing monomers and acrylic esters, cross-linked copolymers of acrylic acid and acrylate esters, heteropolysaccharide gums and crystalline long chain acyl derivatives, and mixtures thereof. The long chain acyl derivative is desirably selected from ethylene glycol stearate, alkanolamides of fatty acids having from 16 to 22 carbon atoms and mixtures thereof. Polyacrylic acid is available commercially as Carbopol 420, Carbopol 488 or Carbopol 493. Polymers of acrylic acid cross-linked with a polyfunctional agent may also be used; they are available commercially as Carbopol 910, Carbopol 934, Carbopol 941 and Carbopol 980. An example of a suitable copolymer of a carboxylic acid containing monomer and acrylic acid esters is Carbopol 1342. All Carbopol (trademark) materials are available from Goodrich. Suitable cross-linked polymers of acrylic acid and acrylate esters are Pemulen TR1 or Pemulen TR2 (both available from Lubrizol). A suitable heteropolysaccharide gum is xanthan gum.

Silicone Conditioning Agents

The hair care compositions of the invention can contain emulsified droplets of a silicone-conditioning agent for enhancing conditioning performance.

Suitable silicones include polydiorganosiloxanes, in particular polydimethylsiloxanes which have the CTFA designation dimethicone. Also suitable for use compositions of the invention (particularly shampoos and conditioners) are polydimethyl siloxanes having hydroxyl end groups, which have the CTFA designation dimethiconol. Also suitable for use in compositions of the invention are silicone gums having a slight degree of cross-linking, as are described for example in WO 96/31188.

Examples of suitable pre-formed silicone emulsions include dimethiconol emulsions DC2-1766, DC2-1784, DC-1785, DC-1786 and DC-1788, all available from Dow Corning. Also suitable are amodimethicone emulsions such as DC2-8177 and DC939 (from Dow Corning) and SME253 (from GE Silicones).

A further preferred class of silicones for inclusion in shampoos and conditioners of the invention have at least one amino functional group.

The total amount of silicone is preferably from 0.01 to 10 of the total composition more preferably from 0.3 to 5, most preferably 0.5 to 3% w/w is a suitable level.

Non-silicone Oily Conditioning Components

The hair care compositions according to the present invention may also comprise a dispersed, non-volatile, water-insoluble oily conditioning agent.

By “insoluble” is meant that the material is not soluble in water (distilled or equivalent) at a concentration of 0.1% w/w at 25° C.

Suitable oily or fatty materials are selected from hydrocarbon oils, fatty esters and mixtures thereof.

Adjuvants

The hair care compositions of the present invention may also contain adjuvants suitable for hair care. Generally such ingredients are included individually at a level of up to 2, preferably up to 1% w/w of the total composition.

Suitable hair care adjuvants, include amino acids, sugars and ceramides. Particularly preferred are anti-dandruff agents, especially those comprising zinc, such as zinc pyrithione (ZnPTO). A further preferred ingredient is climbazole.

Styling Polymers

The hair styling polymer, if present, is preferably present in the hair care compositions of the invention in an amount of from 0.001 to 10, more preferably from 0.1 to 10 by weight, such as from 1 to 8% w/w.

Hair styling polymers are well known. Suitable hair styling polymers include commercially available polymers that contain moieties that render the polymers cationic, anionic, amphoteric or nonionic in nature. Suitable hair styling polymers include, for example, block and graft copolymers. The polymers may be synthetic or naturally derived.

Surfactants

Shampoo compositions preferably comprise one or more cleansing surfactants, which are cosmetically acceptable and suitable for topical application to the hair. Further surfactants may be present as emulsifiers.

Suitable cleansing surfactants are selected from anionic, amphoteric and zwitterionic surfactants and mixtures thereof. The cleansing surfactant may be the same surfactant as the emulsifier, or may be different.

Anionic Cleansing Surfactant

Shampoo compositions according to the invention will typically comprise one or more anionic cleansing surfactants, which are cosmetically acceptable and suitable for topical application to the hair.

Examples of suitable anionic cleansing surfactants are the alkyl sulphates, alkyl ether sulphates, alkaryl sulphonates, alkanoyl isethionates, alkyl succinates, alkyl sulphosuccinates, N-alkyl sarcosinates, alkyl phosphates, alkyl ether phosphates, alkyl ether carboxylates and alpha-olefin sulphonates, especially their sodium, magnesium, ammonium and mono-, di- and triethanolamine salts.

Typical anionic cleansing surfactants for use in shampoo compositions of the invention include sodium oleyl sulpho succinate, ammonium lauryl sulphosuccinate, ammonium lauryl sulphate, sodium cocoyl isethionate, sodium lauryl isethionate and sodium N-lauryl sarcosinate. The most preferred anionic surfactants are sodium lauryl sulphate, sodium lauryl ether sulphate (n)EO (where n ranges from 1 to 3), ammonium lauryl sulphate and ammonium lauryl ether sulphate (n)EO (where n ranges from 1 to 3).

The total amount of anionic cleansing surfactant in shampoo compositions of the invention is generally from 5 to 30, preferably from 6 to 20, more preferably from 8 to 16% w/w.

Co-surfactant

The shampoo composition can optionally include co-surfactants, preferably an amphoteric or zwitterionic surfactant, which can be included in an amount ranging from 0 to about 8, preferably from 1 to 4% w/w.

Examples of amphoteric and zwitterionic surfactants include, alkyl betaines, alkyl amidopropyl betaines, alkyl sulphobetaines (sultaines), alkyl glycinates, alkyl carboxyglycinates, alkyl amphopropionates, alkylamphoglycinates, alkyl amidopropyl hydroxysultaines, acyl taurates and acyl glutamates, wherein the alkyl and acyl groups have from 8 to 19 carbon atoms. Typical amphoteric and zwitterionic surfactants for use in shampoo compositions of the invention include lauryl amine oxide, cocodimethyl sulphopropyl betaine and preferably lauryl betaine, cocamidopropyl betaine and sodium cocamphopropionate.

Another preferred co-surfactant is a nonionic surfactant, which can be included in an amount ranging from 0 to 8, preferably from 2 to 5% w/w.

For example, representative nonionic surfactants that can be included in shampoo compositions of the invention include condensation products of aliphatic (C8-C18) primary or secondary linear or branched chain alcohols or phenols with alkylene oxides, usually ethylene oxide and generally having from 6 to 30 ethylene oxide groups.

Further nonionic surfactants which can be included in shampoo compositions of the invention are the alkyl polyglycosides (APGs). Typically, an APG is one which comprises an alkyl group connected (optionally via a bridging group) to a block of one or more glycosyl groups. Preferred APGs are defined by the following formula:

RO-(G)n

wherein R is a branched or straight chain C5 to C20 alkyl or alkenyl group, G is a saccharide group and n is from 1 to 10.

The shampoo composition can also optionally include one or more cationic co-surfactants included in an amount ranging from 0.01 to 10, more preferably from 0.05 to 5, most preferably from 0.05 to 2% w/w. Useful cationic surfactants are described hereinbelow in relation to conditioner compositions.

The total amount of surfactant (including any co-surfactant, and/or any emulsifier) in shampoo compositions of the invention is generally from 5 to 50, preferably from 5 to 30, more preferably from 10 to 25% w/w.

Cationic Surfactant or Polymer

A cationic surfactant is a preferred ingredient in compositions of the invention for enhancing conditioning performance.

Suitable cationic conditioning surfactants include cetyltrimethylammonium chloride, behenyltrimethylammonium chloride, cetylpyridinium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, dihydrogenated tallow dimethyl ammonium chloride (e.g., Arquad 2HT/75 from Akzo Nobel), cocotrimethylammonium chloride, PEG-2-oleammonium chloride and the corresponding hydroxides thereof, Quaternium-5, Quaternium-31, Quaternium-18 and mixtures thereof.

Another example of a class of suitable cationic conditioning surfactants either alone or in admixture with one or more other cationic conditioning surfactants, is a combination of an amidoamine and an acid. Preferred amidoamines useful herein include stearamido-propyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylmine, behenamidoethyldiethylamine, behenamidoethyldimethylamine, arachidamidopropyldimethylamine, arachidamidopropyldiethylamine, arachid-amidoethyldiethylamine, arachidamidoethyldimethylamine, and mixtures thereof. The acid may be any organic or mineral acid which is capable of protonating the amidoamine in the hair care composition thus forming a tertiary amine salt which is in effect is a non-permanent quaternary ammonium or pseudo-quaternary ammonium cationic surfactant. Suitable acids useful herein include hydrochloric acid, acetic acid, tartaric acid, fumaric acid, lactic acid, malic acid, succinic acid, and mixtures thereof. Suitably, the acid is included in a sufficient amount to protonate all the amidoamine present.

Cationic polymers are preferred ingredients for enhancing conditioning performance. Suitable cationic polymers may be homopolymers which are cationically substituted or may be formed from two or more types of monomers. The weight average (Mw) molecular weight of the polymers will generally be between 100 000 and 2 million daltons. Cationic polymer will generally be present in a shampoo composition at levels of from 0.01 to 5%, preferably from 0.05 to 1%, more preferably from 0.08 to 0.5% w/w of the composition. Suitable cationic polymers include, for example, cationic polysaccharide polymers, copolymers of vinyl monomers having cationic amine or quaternary ammonium functionalities with water soluble spacer monomers such as (meth)acrylamide, alkyl and dialkyl (meth)acrylamides, alkyl (meth)acrylate, vinyl caprolactone and vinyl pyrrolidine. The alkyl and dialkyl substituted monomers preferably have C1-C7 alkyl groups, more preferably C1-3 alkyl groups. Other suitable spacers include vinyl esters, vinyl alcohol, maleic anhydride, propylene glycol and ethylene glycol.

The cationic surfactant or polymer will generally be present in compositions of the invention at levels of from 0.01 to 5, preferably from 0.05 to 1, more preferably from 0.08 to 0.5% w/w.

The combined use of fatty alcohols and cationic surfactants in conditioning compositions is believed to be especially advantageous, because this leads to the formation of a lamellar phase, in which the cationic surfactant is dispersed. The weight ratio of cationic surfactant to fatty alcohol is suitably from 1:1 to 1:10, preferably from 1:1.5 to 1:8, optimally from 1:2 to 1:5. If the weight ratio of cationic surfactant to fatty alcohol is too high, this can lead to eye irritancy from the composition. If it is too low, it can make the hair feel squeaky for some consumers.

Optional Ingredients

A hair care compositions of the invention may contain other ingredients for enhancing performance and/or consumer acceptability. Such ingredients include fragrance, dyes and pigments, pH adjusting agents, pearlescers or opacifiers, viscosity modifiers, and preservatives or antimicrobials. Each of these ingredients will be present in an amount effective to accomplish its purpose. Generally these optional ingredients are included individually at a level of up to 5% w/w of the composition.

Hair care compositions according to the invention are produced using methods known to the person skilled in the art.

The following are examples of formulations according to the invention.

### EXAMPLE 1

### A Hair Growth Slution Composition According to the Invention (% Weight)

| Name | Trade Name | Function | % |
|---|---|---|---|
| Ethanol | Ethanol | Carrier | 45.00 |
| Propylene glycol | Propylene glycol | Carrier | 37.80 |
| Glabranin | | Active | 0.1 |
| Isopropyl myristate | Estol 1514 | Carrier | 10.00 |
| Hydroxypropylcellulose | Klucel M | Thickener | 1.00 |
| DMDM Hydantoin | Glydant | Preservative | 0.20 |
| Water | | | to 100 |

### EXAMPLE 2

### A Hair Growth Lotion Composition According to the Invention (% weight)

| Name | Trade Name | Function | % |
|---|---|---|---|
| DI Water | Deionized water | Initial charge (1) | 55 |
| Methylparaben | Nipagin M | Preservative | 0.3 |
| Behentrimonium | Genammin BTLF | Hair conditioner | 0.31 |
| chloride (70%) | | agent, anti-static | |
| | | agent | |
| Cetearyl alcohol | Hydrenol MY | Emulsifying agent | 0.5 |
| Isopropyl myristate | IPM, Estol 1514 | Conditioning agent | 2 |
| Glabranin | | | 0.1 |
| Glycerin USP | Glycerin 99.7% | Humectant | 1 |
| DI Water | Deionized water | Flush water (2) | 0.1 |
| Perfume | | | 0.4 |
| DI Water | Deionized water | Quench water (3) | To 100 |
| Cyclopentasiloxane | DC(R) 245 Fluid | Conditioning agent | 0.5 |
| 99% | | | |
| DMDM Hydantoin | Glydant, | Preservative | 0.2 |
| | Nipaguard | | |
| | DMDMH | | |
| Polyquat 37 | Salcare SC96 | Viscosity modifier | 0.9 |

### EXAMPLE 3

### In-Vitro Increase in Fibroblast Cell Growth with Glabranin

The assay described hereinbelow looks at the effect of an active on potentiation of cell growth in a low nutrient environment, ie at the effects of the active on cell growth factors which are mitogenic, rather than on the identification of an active which itself is mitogenic.

Glabranin (TimTec) was added in 50 μL of dimethylsulphoxide (DMSO) and vortexed to dissolve. 5 mL of the glabranin in DMSO was added to Dulbeccos Minimum Essential Medium (DMEM) containing 2% v/v foetal calf serum (FCS) to give a 10 μg/mL glabranin solution. Serial dilutions were prepared using DMSO/DMEM/FCS as above. 1 mM minoxidil in DMSO/DMEM/FCS as above was used as a positive control. A DMSO control in DMEM containing 2% v/v FCS was also prepared.

Fibroblast cells from the NIH3T3D4 cell line (European collection of animal cell cultures, Health Protection Agency Porton Down, England) were cultured in DMEM media in the absence of antibiotics. The cells were removed using 10 mL of 0.25% w/w trypsin and 0.038% w/v EDTA in Hank's medium for 5 minutes, 10 mL DMEM containing 10% v/v FCS was added and the cells were spun down at 1000 rpm for 2 minutes. After removing the medium the cells were washed in 10 mL of DMEM containing 10% v/v FCS and spun at 1000 rpm for 2 minutes. The cells were then resuspended in 10 mL of DMEM containing 10% v/v FCS and counted using a cell counting chamber. A suspension of cells containing 5000 cells per 100 μL (50,000 cells per mL) was prepared and 100 μL of the cell suspension was plated out into a 96 well plate. The cells were allowed to adhere overnight in an incubator at 37 degrees centigrade in an atmosphere of 5% v/v CO₂ in air. After washing the wells thrice with DMEM (without FCS), 100 μL of the test solution (containing 1, 0.1 and 0.01 pg glabranin per mL DMSO/DMEM/FCS with or without 10 μM glibenclamide (a potassium ion channel blocker) and 0.1 mM tetraethylammonium (TEA is a potassium ion channel blocker which directly binds to the channel) was added to each of eight wells. Fresh media containing glabranin was added every day for 5 days.

Cell numbers were determined after 5 days in test medium using a colourimetric method (Promega CellTiter Reagent from Promega). 20 μL Reagent was added to each well and the plate incubated as before for one hour. The absorbance was read at 450 nm and the results are shown in table 1.

TABLE 1
The effect of glabranin on fibroblast cell growth and potassium channel opening (the results represent the mean with the SD (standard
deviation) for 8 replicates; glabranin and inhibitor combinations that are significantly different from the same glabranin only concentrations
as measured by the Students t-test are shown as *P < 6 × 10⁻¹⁰, **P < 7 × 10⁻⁹, ***P < 3 × 10⁻⁵).

| | Controls | | | glabranin treatment | | | glabranin + inhibitor treatment | | |
|---|---|---|---|---|---|---|---|---|---|
| | | | 2% FCS/ | concentration | | | concentration (2% FCS/DMSO) | | |
| Cell | | | DMSO + | (2% FCS/DMSO) | | | 0.01 | 0.1 | 1 |
| culture | 10% | 2% FCS/ | Minoxidil | 0.01 | 0.1 | 1 | μg/ml + | μg/ml + | μg/ml + |
| conditions> | FCS | DMSO | (75 μM) | μg/ml | μg/ml | μg/ml | inhibitor | inhibitor | inhibitor |
| Data | 0.48 ± 0.04 | 0.22 ± 0.04 | 0.36 ± 0.04 | 0.34 ± 0.02 | 0.34 ± 0.02 | 0.31 ± 0.02 | 0.22* ± 0.01 | 0.22** ± 0.01 | 0.17*** ± 0.03 |
| (absor- | | | | | | | | | |
| bance at | | | | | | | | | |
| 450 nM)> | | | | | | | | | |

From table 1, it is apparent that glabranin is a potassium channel opener and this leads to fibroblast cell growth.

1. A hair care composition comprising glabranin or a derivative thereof.

2. A hair care composition according to claim 1 wherein the glabranin or a derivative thereof is in the form of an extract of any one of the group consisting of a leaf of Glycyrrhiza glabra, a seed of Annona glabra and Tephrosia madrensis.

3. A hair care composition according to claim 1 or claim 2, wherein the glabranin or a derivative thereof is in the weight range 0.00001 to 30%, preferably 0.0001 to 15%, most preferably 0.001 to 5% by weight of the composition.

4. A hair care composition according to any one of the preceding claims, wherein the hair care composition additionally comprises a further hair growth active.

5. A hair care composition according to any one of the preceding claims in the form of a shampoo or a conditioner.

6. Glabranin or a derivative thereof for use in increasing the proliferation of cells within a hair follicle.

7. A cosmetic method for increasing hair fibre diameter, the method comprising the step of applying the hair care composition according to any one of claims 1 to 5 to a scalp or hair.

8. A cosmetic method for stimulating hair growth, the method comprising the step of applying the hair care composition according to any one of claims 1 to 5 to a scalp or hair.

9. A cosmetic method for retaining hair or reducing hair loss, the method the step of comprising applying the hair care composition according to any one of claims 1 to 5 to a scalp or hair.

10. A cosmetic method for stimulating anagen induction, the method comprising the step of applying the hair care composition according to any one of claims 1 to 5 to a scalp or hair.

11. A cosmetic method for increasing hair fibre density, the method comprising the step of applying the hair care composition according to any one of claims 1 to 5 to a scalp or hair.

12. A cosmetic method for preventing or treating alopecia, the method comprising the step of applying the hair care composition according to any one of claims 1 to 5 to a scalp or hair.
